(19) United States
(12) Patent Application Publication
(10) Pub. No.: US 2009/0170857 A1
(43) Pub. Date: Jul. 2, 2009

(54) Method for healing a wound using a direct vasodilator

(76) Inventors: Matthew H. Kopacki, Ringwood, NJ (US); Michael J. Torsiello, Pleasant Mount, PA (US)

Correspondence Address: King & Partners, PLC, 170 College Avenue, SUITE 230, HOLLAND, MI, 49423, US

(21) Appl. No.: 12/380,102

(22) Filed: Feb. 24, 2009

Related U.S. Application Data
(63) Continuation-in-part of application No. 11/978,293, filed on Oct. 29, 2007.
(60) Provisional application No. 60/854,805, filed on Oct. 27, 2006.

(51) Int. Cl. A61K 31/502; A61K 31/506; A61K 31/138; A61K 31/472; A61P 17/02

(52) U.S. Cl. 514/248; 514/275; 514/651; 514/307; 514/263.31

(57) ABSTRACT

A method for healing a wound comprising the step of topically administering a wound healing composition to a wounded area, wherein the wound healing composition comprises a first medicament characterized as a direct vasodilator and pharmaceutically acceptable salts thereof.

### CROSS REFERENCE TO RELATED APPLICATION(S)

This application is a continuation-in-part of U.S. application Ser. No. 11/978,293, filed Oct. 29, 2007, entitled “METHOD FOR HEALING A WOUND,” which claims the benefit of U.S. Provisional Application Ser. No. 60/854,805, filed Oct. 27, 2006, entitled “METHOD FOR HEALING A WOUND,” which are hereby incorporated herein by reference in their entirety—including all references cited therein.

### BACKGROUND OF THE INVENTION

1. Field of the Invention

The present invention relates in general to a method for healing a wound and, more particularly, to a method for healing a wound via localized, topical application of one or more direct vasodilators.

2. Background Art

In today's society, many people suffer from difficult-to-heal wounds that are refractory to conventional forms of treatment. Such difficult-to-heal wounds may include diabetic skin sores, pressure sores, vessel disease wounds, surgery wound breakdown, spinal injury wounds, and chemical wounds—just to name a few.

To be sure, the human body's healing process is very complex and requires several steps. According to The Wound Care Information Center, sponsored by the Wound Care Center at Presbyterian Hospital of Dallas, normal healing requires that cells proliferate and divide, thereby releasing growth factors. In turn, new blood vessels are produced, a collagen matrix is formed, and remodeling occurs. Each step requires appropriate substrates and nutritional elements to be present and available. However, for some patients, certain conditions alter this course, thereby disrupting the healing process. In such cases, the wound can become chronic. In America alone, approximately five million (5,000,000) people are battling chronic open sores which may become seriously infected, gangrenous, and may eventually require amputation.

In addition, the Wound Care Information Center estimates that there are approximately sixteen million (16,000,000) diabetics in America. Diabetes causes microangiopathic changes in, among other places, the foot, which is a common site for non-healing wounds. It is not unlikely for diabetic patients to have to undergo surgical amputation as a result of a non-healing wound and must then face a lifetime of costly rehabilitation, and permanently reduced mobility and independence.

Other conditions which can lead to the development of non-healing wounds include peripheral vascular disease, arterial or venous ulcers, traumatic injury, complications following surgery, rheumatoid arthritis, congestive heart failure, lymphedema, and other conditions which compromise circulation. In addition, local factors such as pressure, infection, or edema, and systemic problems which leave patients immunocompromised, such as collagen vascular disease, acquired immunodeficiency syndrome, rheumatoid arthritis, or diabetes mellitus, can impair normal healing. Furthermore, some medications can suppress the body's healing process and inadequate large-vessel perfusion and oxygenation impedes healing by reducing the oxygen supply to the damaged tissue.

At this time, wound treatment programs and intervention include conventional and advanced wound dressings, removal of unhealthy tissue, bioengineered tissue, hyperbaric (high-pressure) oxygen treatment, growth factors (isolated, concentrated substances that are applied topically to the wound to stimulate healing), antibiotic therapy, nutrition counseling, education and prevention, surgery, physical therapy, and protective footwear, among others.

Patients suffering from such wounds and/or chronic open sores typically seek specialized professional help after their wounds have not healed during months of standard wound treatment. However, even the most advanced methods for healing wounds can take several additional months and are not always successful.

It is therefore an object of the present invention, to provide a method for healing a wound via localized, topical application of a first medicament (i.e. a direct vasodilator) to remedy and/or minimize the aforementioned problems and/or complications associated with healing a wound.

### SUMMARY OF THE INVENTION

The present invention is directed to a method for healing a wound comprising the step of: (a) topically administering a wound healing composition to a wounded area, wherein the wound healing composition comprises a first medicament characterized as a direct vasodilator and pharmaceutically acceptable salts thereof.

In a preferred embodiment of the present invention, the step of topically administering a wound healing composition comprises the step of topically administering a first medicament which comprises at least one of minoxidil, hydralazine, isoxsuprine, papaverine, and pharmaceutically acceptable salts thereof.

In another preferred embodiment of the present invention, the step of topically administering a wound healing composition comprises the step of topically administering a first medicament represented by one or more of the following chemical structures:

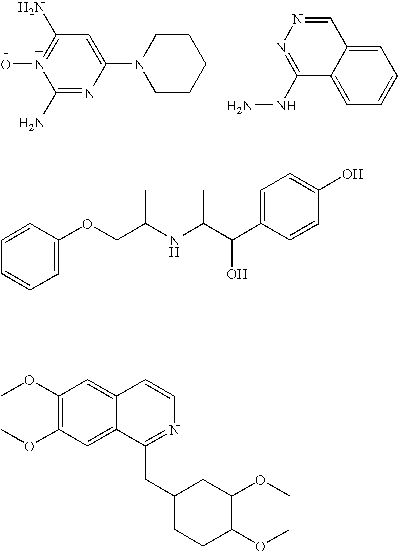

and pharmaceutically acceptable salts thereof.

In yet another preferred embodiment of the present invention, the step of topically administering a wound healing composition comprises the step of topically administering a wound healing composition which further comprises a second medicament characterized as a hemorrheologic agent.

In such an embodiment the hemorrheologic agent preferably comprises a second medicament represented by the following chemical structure:

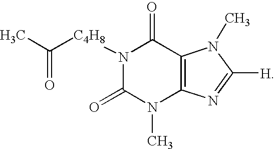

and pharmaceutically acceptable salts thereof.

In another aspect of the present invention, the step of topically administering a wound healing composition comprises the step of topically administering a wound healing composition which further comprises a dermal penetrating agent.

In this embodiment the dermal penetrating agent is preferably represented by the following chemical structure:

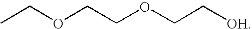

In another preferred embodiment of the present invention, the step of topically administering a wound healing composition comprises the step of topically administering a wound -healing composition which comprises: a first medicament comprising 2,6-diamino-4-(piperidin-1-yl)pyrimidine 1-oxide and pharmaceutically acceptable salts thereof; a second medicament comprising 1-(5-oxohexyl)-3,7-dimethylxanthine and pharmaceutically acceptable salts thereof; and a dermal penetrating agent comprising 2-(2-ethoxy-ethoxy)-ethanol and pharmaceutically acceptable salts thereof.

The present invention is also directed to a method for healing a wound, comprising the step of: (a) topically administering a wound healing composition to a wounded area, wherein the wound healing composition comprises: (1) a first medicament characterized as a direct vasodilator and pharmaceutically acceptable salts thereof; (2) a second medicament characterized as a hemorrheologic agent and pharmaceutically acceptable salts thereof; and (3) a dermal penetrating agent.

In the foregoing embodiments the step of topically administering a wound healing composition preferably comprises the step of topically administering an effective amount of a wound healing composition to the wounded area.

### DETAILED DESCRIPTION OF THE INVENTION

While this invention is susceptible of embodiment in many different forms, there will herein be described in detail several specific embodiments with the understanding that the present disclosure is to be considered as an exemplification of the principles of the invention and is not intended to limit the invention to the embodiments illustrated.

In accordance with the present invention, a method for healing a wound is disclosed which comprises the steps of topically administering a wound healing composition to a wounded area, wherein the wound healing composition comprises: a first medicament characterized as a direct vasodilator and pharmaceutically acceptable salts thereof; optionally a second medicament characterized as a hemorrheologic agent and pharmaceutically acceptable salts thereof; and optionally a dermal penetrating agent and pharmaceutically acceptable salts thereof.

It will be understood that regardless of it ordinary meaning the term “wounded area” will be defined herein as an area having an open and/or closed injury as well as any surrounding periphery that may, but not necessarily, involve a laceration or breaking of a membrane (e.g. skin) and may, but not necessarily, involve damage to underlying tissue. Indeed, in accordance with the present invention, many times a wound healing composition is associated with and/or applied to the surrounding periphery of the wound without actually applying the same to the open, closed, and/or irritated portion of the wound.

In accordance with the present invention, the first medicament comprises one or more direct vasodilator(s), including, for example, minoxidil (Rogaine, Regaine), hydralazine (Apresoline), isoxsuprine, and papaverine—just to name a few. It will be understood that many of the above-identified direct vasodilators are readily available for any one of a number of common commercial sources.

In one aspect of the present invention, the first medicament (characterized as a direct vasodilator) is preferably represented by one or more of the following chemical structures:

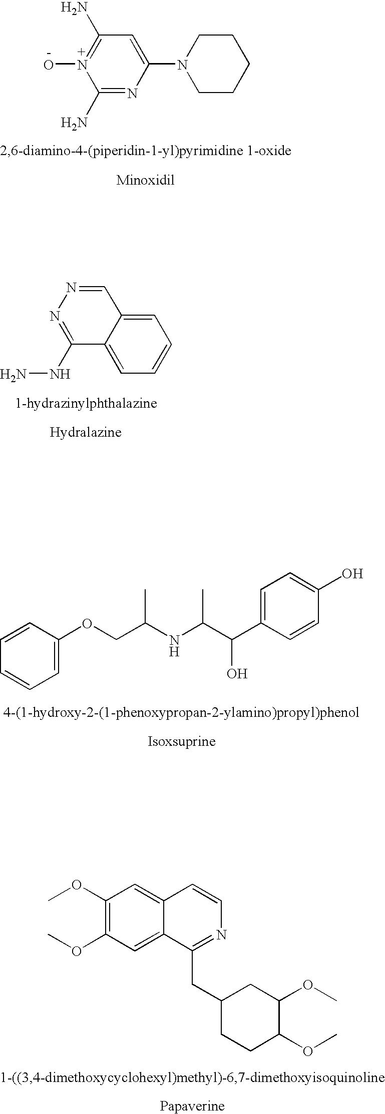

and pharmaceutically acceptable salts thereof.

It will be understood that an “effective amount” of the above-identified first medicaments is an amount that facilitates healing of a wound, and can be administered, via any one of a number of conventional means, to a patient/subject. Preferably, the effective dose ranges in concentration from approximately 0.05% (wt.) to approximately 40% (wt.) approximately q.d.-q.i.d., and more preferably ranges in concentration from approximately 0.1% (wt.) to approximately 20% (wt.) approximately q.d.-q.i.d. However, the effective amount will vary depending upon many factors including, for example, the size and type of wound being treated.

For purposes of the present disclosure, the second medicament (characterized as a hemorrheologic agent) is preferably represented by the following chemical structure:

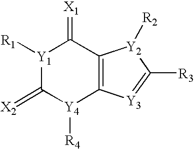

wherein R₁₋₄ are the same or different and comprise H, a hydroxy group, a primary, secondary, or tertiary amine, a straight or branched alkyl, cycloalkyl, polycycloalkyl, heterocycloalkyl, aryl, alkaryl, aralkyl, alkoxy, alkenyl, alkynyl, or carbonyl group containing approximately 1 to approximately 50 carbon atom(s), a silyl or siloxyl group containing approximately 1 to approximately 50 silicon atom(s), and combinations thereof; wherein X₁₋₂ are the same or different and comprise oxygen, sulfur, or selenium; and wherein Y₁₋₄ are the same or different and comprise nitrogen or phosphorus.

Preferably, the second medicament is represented by the following chemical structure:

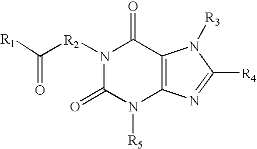

wherein R₁₋₅ are the same or different and comprise H, a hydroxy group, a primary, secondary, or tertiary amine, a straight or branched alkyl, cycloalkyl, polycycloalkyl, heterocycloalkyl, aryl, alkaryl, aralkyl, alkoxy, alkenyl, alkynyl, or carbonyl group containing approximately 1 to approximately 50 carbon atom(s), a silyl or siloxyl group containing approximately 1 to approximately 50 silicon atom(s), and combinations thereof. One specific example includes the following chemical structure:

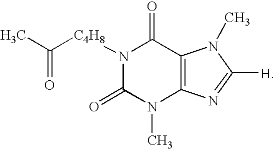

For purposes of clarity, and in an attempt to eliminate any potential ambiguity associated with the nomenclature of the above-identified second medicament, it will be understood that a specific medicament provided herein above is defined as 1-(5-oxohexyl)-3,7-dimethylxanthine, which is commercially available from Aventis Pharmaceuticals.

It will be understood that an “effective amount” of the above-identified second medicaments is an amount that facilitates healing of a wound, and can be administered, via any one of a number of conventional means, to a patient/subject. Preferably, the effective dose ranges in concentration from approximately 0.5% (wt.) to approximately 40% (wt.) approximately q.d.-q.i.d., and more preferably ranges in concentration from approximately 5% (wt.) to approximately 15% (wt.) approximately q.d.-q.i.d. However, the effective amount will vary depending upon many factors including, once again for example, the size and type of wound being treated.

In accordance with the present invention, the dermal penetrating agent is preferably represented by the following chemical structure:
R₁—X₁—R₂—X₂—R₃—X₃—R₄
   wherein R₁₋₄ are the same or different and comprise H, a hydroxy group, a primary, secondary, or tertiary amine, a straight or branched alkyl, cycloalkyl, polycycloalkyl, heterocycloalkyl, aryl, alkaryl, aralkyl, alkoxy, alkenyl, alkynyl, or carbonyl group containing approximately 1 to approximately 50 carbon atom(s), a silyl or siloxyl group containing approximately 1 to approximately 50 silicon atom(s), and combinations thereof; and wherein X₁₋₃ are the same or different and comprise oxygen, sulfur, or selenium.

Preferably, the dermal penetrating agent is represented by the following chemical structure:

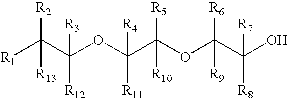

wherein R₁₋₁₃ are the same or different and comprise H, a hydroxy group, a primary, secondary, or tertiary amine, a straight or branched alkyl, cycloalkyl, polycycloalkyl, heterocycloalkyl, aryl, alkaryl, aralkyl, alkoxy, alkenyl, alkynyl, or carbonyl group containing approximately 1 to approximately 50 carbon atom(s), a silyl or siloxyl group containing approximately 1 to approximately 50 silicon atom(s), and combinations thereof. A specific example includes the following chemical structure:

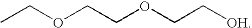

For purposes of clarity, and in an attempt to eliminate any potential ambiguity associated with the nomenclature of the above-identified dermal penetrating agents, it will be understood that a specific dermal penetrating agent provided herein above is defined as 2-(2-ethoxy-ethoxy)-ethanol, which is commercially available from Pfaltz and Bauer.

It will be understood that an “effective amount” of the above-identified dermal penetrating agents is an amount that facilitates healing of a wound, and can be administered, via any one of a number of conventional means, to a patient/subject. Preferably, the effective dose ranges in concentration from approximately 0.5% (wt.) to approximately 60% (wt.) approximately q.d.-q.i.d., and more preferably ranges in concentration from approximately 5% (wt.) to approximately 20% (wt.) approximately q.d.-q.i.d. However, the effective amount will vary depending upon many factors including, as mentioned herein, the size and type of wound being treated.

In operation a topical formulation (5 mg/ml) is prepared comprising approximately 0.5% (wt.) of minoxidil (i.e. 2,6-diamino-4-(piperidin-1-yl)pyrimidine 1-oxide), 10% (wt.) pentoxifylline (i.e. 1-(5-oxohexyl)-3,7-dimethylxanthine), 12.5% (wt.) ethoxy diglycol (i.e. 2-(2-ethoxy-ethoxy)-ethanol), 22% (wt.) lecithin/isopropyl palmitate, and pluronic F-127 (to 100%). The above-identified composition is topically administered to the outer periphery of a subject having a wounded area approximately q.d.-q.i.d. It will be understood that formulations void of a hemorrheologic agent and/or a dermal penetrating agent are suitable for use in accordance with the present invention.

The foregoing description merely explains and illustrates the invention and the invention is not limited thereto except insofar as the appended claims are so limited, as those skilled in the art who have the disclosure before them will be able to make modifications without departing the scope of the invention.

What is claimed is:

1. A method for healing a wound, comprising the step of:
topically administering a wound healing composition to a wounded area, wherein the wound healing composition comprises a first medicament characterized as a direct vasodilator and pharmaceutically acceptable salts thereof.

2. The method for healing a wound according to claim 1, wherein the step of topically administering a wound healing composition comprises the step of topically administering an effective amount of a wound healing composition to the wounded area.

3. The method for healing a wound according to claim 1, wherein the step of topically administering a wound healing composition comprises the step of topically administering a first medicament which comprises at least one of minoxidil, hydralazine, isoxsuprine, papaverine, and pharmaceutically acceptable salts thereof.

4. The method for healing a wound according to claim 3, wherein the step of topically administering a wound healing composition comprises the step of topically administering an effective amount of a wound healing composition to the wounded area.

5. The method for healing a wound according to claim 1, wherein the step of topically administering a wound healing composition comprises the step of topically administering a first medicament represented by the following chemical structure: 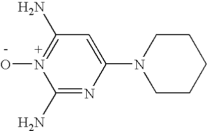
and pharmaceutically acceptable salts thereof.

6. The method for healing a wound according to claim 1, wherein the step of topically administering a wound healing composition comprises the step of topically administering a first medicament represented by the following chemical structure: 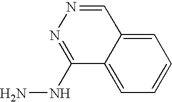
and pharmaceutically acceptable salts thereof.

7. The method for healing a wound according to claim 1, wherein the step of topically administering a wound healing composition comprises the step of topically administering a first medicament represented by the following chemical structure: 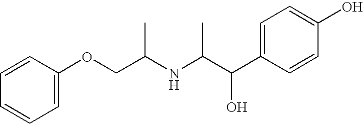
and pharmaceutically acceptable salts thereof.

8. The method for healing a wound according to claim 1, wherein the step of topically administering a wound healing composition comprises the step of topically administering a first medicament represented by the following chemical structure: 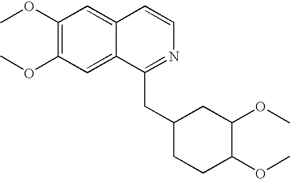
and pharmaceutically acceptable salts thereof.

9. The method for healing a wound according to claim 1, wherein the step of topically administering a wound healing composition comprises the step of topically administering a wound healing composition which further comprises a second medicament characterized as a hemorrheologic agent.

10. The method for healing a wound according to claim 9, wherein the step of topically administering a wound healing composition comprises the step of topically administering an effective amount of a wound healing composition to the wounded area.

11. The method for healing a wound according to claim 1, wherein the step of topically administering a wound healing composition comprises the step of topically administering a wound healing composition which further comprises a second medicament represented by the following chemical structure: 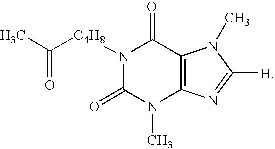
and pharmaceutically acceptable salts thereof.

12. The method for healing a wound according to claim 1, wherein the step of topically administering a wound healing composition comprises the step of topically administering a wound healing composition which further comprises a dermal penetrating agent.

13. The method for healing a wound according to claim 12, wherein the step of topically administering a wound healing composition comprises the step of topically administering an effective amount of a wound healing composition to the wounded area.

14. The method for healing a wound according to claim 1, wherein the step of topically administering a wound healing composition comprises the step of topically administering a wound healing composition which further comprises a dermal penetrating agent represented by the following chemical structure:

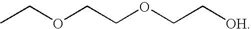

15. The method for healing a wound according to claim 1, wherein the step of topically administering a wound healing composition comprises the step of topically administering a wound healing composition which comprises: a first medicament comprising 2,6-diamino-4-(piperidin-1-yl)pyrimidine 1-oxide and pharmaceutically acceptable salts thereof; a second medicament comprising 1-(5-oxohexyl)-3,7-dimethylxanthine and pharmaceutically acceptable salts thereof; and a dermal penetrating agent comprising 2-(2-ethoxy-ethoxy)-ethanol and pharmaceutically acceptable salts thereof.

16. The method for healing a wound according to claim 15, wherein the step of topically administering a wound healing composition comprises the step of topically administering an effective amount of a wound healing composition to the wounded area.

17. A method for healing a wound, comprising the steps of:
topically administering a wound healing composition to a wounded area, wherein the wound healing composition comprises: a first medicament characterized as a direct vasodilator and pharmaceutically acceptable salts thereof; a second medicament characterized as a hemorrheologic agent and pharmaceutically acceptable salts thereof; and a dermal penetrating agent.

18. The method for healing a wound according to claim 17, wherein the step of topically administering a wound healing composition comprises the step of topically administering an effective amount of a wound healing composition to the wounded area.
